(19) United States
(12) Patent Application Publication
(10) Pub. No.: US 2005/0014844 A1
(43) Pub. Date: Jan. 20, 2005

(54) Ambroxol for the treatment of acute pain

(75) Inventors: Wolfram Gaida, Ingelheim am Rhein (DE); Klaus Klinder, Oggelshausen (DE); Thomas Weiser, Biberach (DE)

Correspondence Address: BOEHRINGER INGELHEIM CORPORATION, 900 RIDGEBURY ROAD, P. O. BOX 368, RIDGEFIELD, CT, 06877, US

(73) Assignee: Boehringer Ingelheim International GmbH, Ingelheim (DE)

(21) Appl. No.: 10/888,185

(22) Filed: Jul. 9, 2004

(30) Foreign Application Priority Data
Jul. 16, 2003 (DE) DE 103 32 486

(51) Int. Cl. A61K 31/137

(52) U.S. Cl. 514/650

(57) ABSTRACT

A method of treating acute pain in a patient in need thereof, the method comprising orally administering to the patient ambroxol or a pharmacologically acceptable salt thereof.

### RELATED APPLICATIONS

This application claims priority to German patent application No. DE 103 32 486.0, filed Jul. 16, 2003, which is hereby incorporated by reference in its entirety.

### FIELD OF THE INVENTION

The invention relates to the use of ambroxol and the pharmacologically acceptable salts thereof for preparing a pharmaceutical composition for the treatment of acute pain.

### BACKGROUND OF THE INVENTION

The active substance ambroxol (trans-4-(2-amino-3,5-dibromobenzylamino)cyclohexanol) is a known local anesthetic, antitussive, and expectorant. In addition, ambroxol's effect as a sodium channel blocker is described in the literature (Society for Neuroscience Abstracts, 2000, Vol. 26, No. 1-2). The potential activity of sodium channel blockers as pain relievers is also known from the prior art (Mao and Chen (2000), Pain 87, 7-17). Although known sodium channel blockers are discussed in the literature for the systemic treatment of acute pain, they are not, however, fundamentally suitable for the systemic treatment of acute pain, because of their range of side-effects, for example. As a rule, they are used only for local, regional, or conductive anesthesia.

Known sodium channel blockers generally act selectively by blocking the sodium channels. In addition, they are not suitable for oral administration in every case and frequently exhibit both cardiovascular and central nervous side effects (Groban (2003), Regional Anesthesia and Pain Medicine 28, 3-11; Webb and Kamali (1998), Pain 76, 357-363).

It is also known from the prior art that calcium channel blockers and AMPA receptor agonists (α-amino-3-hydroxy-5-methyl4-isoxazolepropionate) exhibit good effects in pain models for acute pain (Sluka (1998), JPET 287, 232-237; Saegusa, Matsuda, and Tanabe (2002), Neurosci Res 43, 1-7; Szekely et al. (1997), Europ J Pharmacol 336, 143-154).

Other active substances for the systemic treatment of acute pain, for example, non-steroidal anti-inflammatory substances (NSAIDs), often display gastrointestinal side effects, and there is even damage to the liver and kidneys in some cases (Epstein (2002), J Hypertens 20 Suppl. 6, 17-23; Boelsterlie (2002), Drug Saf 25, 633-648).

The aim of the present invention is to provide a well-tolerated active substance for the systemic treatment of acute pain. In particular, the active substance provided should be suitable for oral administration and hence have good bioavailability.

### DESCRIPTION OF THE INVENTION

Surprisingly, ambroxol shows a very good activity in the treatment of acute pain. At a pharmacologically effective dose there are no central nervous or cardiovascular side effects. Surprisingly, ambroxol also exhibits very good effects as a calcium channel blocker and as an AMPA receptor antagonist, which give rise to a potent antinociceptive activity.

The invention therefore relates to the use of ambroxol or one of the pharmacologically acceptable salts thereof for preparing a pharmaceutical composition for the oral treatment of acute pain.

Preferably ambroxol or one of the pharmacologically acceptable salts thereof is used to prepare a pharmaceutical composition for the treatment of operative pain.

Also preferred is the use of ambroxol or one of the pharmacologically acceptable salts thereof for preparing a pharmaceutical composition for the treatment of acute pain in herpes zoster.

It is particularly preferred to use ambroxol or one of the pharmacologically acceptable salts thereof for preparing a pharmaceutical composition for the treatment of toothache.

It is also particularly preferred to use ambroxol or one of the pharmacologically acceptable salts thereof for preparing a pharmaceutical composition for the treatment of pain after trauma.

Particularly preferred is the use of ambroxol or one of the pharmacologically acceptable salts thereof for preparing a pharmaceutical composition for the treatment of pain after burns.

It is also particularly preferred to use ambroxol or one of the pharmacologically acceptable salts thereof for preparing a pharmaceutical composition for the treatment of pain after stroke or cardiac infarct.

It is also preferred to use ambroxol or one of the pharmacologically acceptable salts thereof for preparing a pharmaceutical composition for the treatment of pain in pancreatitis.

It is also preferred to use ambroxol or one of the pharmacologically acceptable salts thereof for preparing a pharmaceutical composition for the treatment of pain in colic.

It is also preferred to use ambroxol or one of the pharmacologically acceptable salts thereof for preparing a pharmaceutical composition for the treatment of pain in cramps.

It is particularly preferred to use ambroxol for preparing a pharmaceutical composition for the treatment of the types of pain described above if they cannot be satisfactorily treated with conventional analgesics, for example, NSAIDs.

The invention further relates to a pharmaceutical composition, particularly a composition for oral administration, containing ambroxol and one or more active substances selected from the group consisting of NSAIDs, for example, salicylic acid derivatives, arylacetic acid or arylpropionic acid derivatives, anthranilic acid derivatives, pyrazolone derivatives, oxicams, opioids, anticonvulsants, local anesthetics, antidepressants, and glutamate receptor antagonists, preferably one or more active substances selected from the group consisting of acetylsalicylic acid, diclofenac, ibuprofen, paracetamol, flufenamic acid, mefenamic acid, indometacin, morphine, pethidine, methadone, fentanyl, buprenorphine, tramadol, gabapentin, pregabalin, carbamazepine, lamotrigin, topiramate, phenytoin, levitiracetam, procaine, lidocaine, mepivacaine, articaine, prilocaine, etidocaine, bupivacaine, ropivacaine, amitryptiline, paroxetine, citalopram, bupropione, duxoletine, ketamine, memantine, 2,3-benzodiazepines, and quinoxaline-diones.

The invention further relates to the use of ambroxol or one of the pharmacologically acceptable salts thereof in combination with one or more other active substances, selected from the group consisting of NSAIDs, opioids, anticonvulsants, local anesthetics, antidepressants, and glutamate receptor antagonists, preferably one or more active substances selected from the group consisting of acetylsalicylic acid, diclofenac, ibuprofen, paracetamol, flufenamic acid, mefenamic acid, indometacin, morphine, pethidine, methadone, fentanyl, buprenorphine, tramadol, gabapentin, pregabalin, carbamazepine, lamotrigin, topiramate, phenytoin, levitiracetam, procaine, lidocaine, mepivacaine, articaine, prilocaine, etidocaine, bupivacaine, ropivacaine, amitryptiline, paroxetine, citalopram, bupropione, duxoletine, ketamine, memantine, 2,3-benzodiazepines, and quinoxaline-diones.

The invention further relates to a pharmaceutical composition, particularly a composition for oral administration, containing ambroxol and one or more active substances selected from the group consisting of calcium channel blockers, preferably N subtype blockers, P/Q subtype blockers, gallopamil, verapamil, diltiazem, nifedipine, or amlodipine, sodium channel blockers, preferably local anesthetics, anticonvulsants, or antiarrhythmics, most preferably mexiletine, nicotine agonists, and alpha-adrenergic agonists, preferably clonidine, moxonidine, or guanfacine.

The invention further relates to the use of ambroxol or one of the pharmacologically acceptable salts thereof in combination with one or more other pain relievers, selected from the group consisting of calcium channel blockers, sodium channel blockers, nicotine agonists, and alpha-adrenergic agonists.

Ambroxol is preferably used to treat patients with acute pain or patients with acute pain combined with other forms of pain, preferably chronic pain, for example, chronic nociceptive or chronic neuropathic pain, particularly chronic neuropathic pain.

The name ambroxol within the scope of the present invention denotes both the base ambroxol, and also the solvates or hydrates thereof, preferably the base ambroxol.

Acids suitable for forming salts of ambroxol are, for example, hydrochloric acid, hydrobromic acid, sulfuric acid, phosphoric acid, nitric acid, oxalic acid, malonic acid, fumaric acid, maleic acid, tartaric acid, citric acid, ascorbic acid, and methanesulfonic acid, preferably hydrochloric acid.

The effects of ambroxol according to the invention will be illustrated by the Examples that follow. These serve merely to illustrate the invention and are not to be regarded as limiting it.

Analgesic substances, such as, e.g., local anesthetics, act by blocking the voltage-dependant sodium channels. Ambroxol inhibits these channels. Unlike the sodium channel blockers described which are in clinical use, ambroxol preferentially inhibits tetrodotoxin-resistant sodium channels in nociceptive C-fiber neurons. C-fiber neurons are essential for the perception of acute pain stimuli, as well as for transmitting the stimulation to higher centers of the brain (Weiser and Wilson (2002), Mol Pharmacol 62, 433-438; ladarola et al. (1998), Brain 121, 931-47).

In neuron cultures from the posterior root ganglia of adult rats, tetrodotoxin-resistant sodium channels were half-maximally inhibited by 35 μM of ambroxol. Tetrodotoxin-sensitive currents were inhibited much less powerfully by this concentration, the IC₅₀ here being more than 100 μM.

Voltage-dependent calcium channels play an important role in the transmission of pain-related excitation in the nervous system. The blocking of these channels has been described as being helpful for the treatment of acute pain (KA Sluka (1998), JPET 287, 232-237; J Devulder et al. (2001), J Pain Sympt Management 22, 622-626). It has been found, surprisingly, that ambroxol also blocks voltage-dependent calcium channels in neuron cultures from rats in concentrations of 10 to 1000 μM. Neurons were dissected from posterior root ganglia of adult rats and placed in short-term culture. The cells were investigated electrophysiologically by the Patch-Clamp method (voltage terminal), and the flow of current through voltage-dependent calcium channels was measured after electrical stimulation (voltage jumps from −80 mV to 0 mV holding potential for 50 ms) in the presence and absence of ambroxol.

Ionotropic glutamate receptors of the AMPA sub-type are also essential for the excitatory neurotransmission. In HEK 293 cells which express heterologously human GluR1/2 receptors, ambroxol surprisingly inhibits glutamate-induced membrane currents in concentrations ranging from 30-1000 μM. The blocking of AMPA receptors has been described as being helpful for the treatment of acute pain (e.g., ji Szekely et al. (1997), EJP 336, 143-154). HEK 293 cells which expressed functionally recombinant human GluR1/2 receptors were electrophysiologically investigated by the Patch-Clamp method (voltage terminal). The administration of 1 mM glutamate (for 1 s at a holding potential of −80 mV) induced membrane currents which were inhibited by the joint administration of ambroxol.

Ambroxol may be used on its own or in combination with other pharmacologically active substances. Suitable preparations include for example tablets, capsules, suppositories, solutions, elixirs, emulsions, ointments, or dispersible powders. Suitable tablets may be obtained, for example, by mixing the active substance(s) with known excipients, for example, inert diluents such as calcium carbonate, calcium phosphate, or lactose, disintegrants such as corn starch or alginic acid, binders such as starch or gelatine, lubricants such as magnesium stearate or talc and/or agents for delaying release, such as carboxymethyl cellulose, cellulose acetate phthalate, or polyvinyl acetate. The tablets may also comprise several layers.

Coated tablets may be prepared accordingly by coating cores produced analogously to the tablets with substances normally used for tablet coatings, for example, collidone or shellac, gum arabic, talc, titanium dioxide, or sugar. To achieve delayed release or prevent incompatibilities the core may also consist of a number of layers. Similarly the tablet coating may consist of a number or layers to achieve delayed release, possibly using the excipients mentioned above for the tablets.

Syrups or elixirs containing the active substances or combinations thereof according to the invention may additionally contain a sweetener such as saccharine, cyclamate, glycerol, or sugar and a flavor enhancer, e.g., a flavoring such as vanillin or orange extract. They may also contain suspension adjuvants or thickeners such as sodium carboxymethyl cellulose, wetting agents such as, for example, condensation products of fatty alcohols with ethylene oxide, or preservatives such as p-hydroxybenzoates.

Solutions for injection are prepared in the usual way, e.g., with the addition of preservatives such as p-hydroxybenzoates, or stabilizers such as alkali metal salts of ethylenediamine tetraacetic acid, and transferred into injection vials or ampoules.

Capsules containing one or more active substances or combinations of active substances may for example be prepared by mixing the active substances with inert carriers such as lactose or sorbitol and packing them into gelatine capsules.

Suitable suppositories may be made, for example, by mixing with carriers provided for this purpose, such as neutral fats or polyethyleneglycol or the derivatives thereof.

A therapeutically effective daily dose is in the range from 30 mg to 4000 mg, preferably 150 mg to 3000 mg, more preferably 350 mg to 2500 mg, and most preferably 500 mg to 2000 mg, in adults.

The Examples which follow illustrate the present invention without restricting its scope:

### EXAMPLES OF PHARMACEUTICAL FORMULATIONS

| A) | Tablets | per tablet |
|---|---|---|
| | ambroxol | 800 mg |
| | lactose | 140 mg |
| | maize starch | 240 mg |
| | polyvinylpyrrolidone | 20 mg |
| | magnesium stearate | 10 mg |

Ambroxol, lactose, and some of the maize starch are mixed together. The mixture is screened, then moistened with a solution of polyvinylpyrrolidone in water, kneaded, wet-granulated, and dried. The granules, the remaining maize starch and the magnesium stearate are screened and mixed together. The mixture is compressed to produce tablets of suitable shape and size.

| B) | Tablets | per tablet |
|---|---|---|
| | ambroxol | 800 mg |
| | maize starch | 190 mg |
| | lactose | 55 mg |
| | microcrystalline cellulose | 35 mg |
| | polyvinylpyrrolidone | 20 mg |
| | sodium-carboxymethyl starch | 30 mg |
| | magnesium stearate | 10 mg |

Ambroxol, some of the corn starch, lactose, microcrystalline cellulose, and polyvinylpyrrolidone are mixed together, the mixture is screened and worked with the remaining corn starch and water to form a granulate which is dried and screened. The sodium-carboxymethyl starch and the magnesium stearate are added and mixed in and the mixture is compressed to form tablets of a suitable size.

| C) | Coated tablets | per coated tablet |
|---|---|---|
| | ambroxol | 500 mg |
| | maize starch | 45 mg |
| | lactose | 30 mg |
| | polyvinylpyrrolidone | 5 mg |
| | magnesium stearate | 5 mg |

Ambroxol, maize starch, lactose, and polyvinylpyrrolidone are thoroughly mixed and moistened with water. The moist mass is pushed through a screen with a 1 mm mesh size, dried at about 45° C. and the granules are then passed through the same screen. After the magnesium stearate has been mixed in, convex tablet cores with a diameter of 11 mm are compressed in a tablet-making machine. The tablet cores thus produced are coated in known manner with a covering consisting essentially of sugar and talc. The finished coated tablets are polished with wax.

| D) | Capsules | per capsule |
|---|---|---|
| | ambroxol | 250 mg |
| | maize starch | 268.5 mg |
| | magnesium stearate | 1.5 mg |

Ambroxol and maize starch are mixed and moistened with water. The moist mass is screened and dried. The dry granules are screened and mixed with magnesium stearate. The finished mixture is packed into size 1 hard gelatine capsules.

| E) | Parenteral Solution | | |
|---|---|---|---|
| | ambroxol | 500 | mg |
| | citric acid monohydrate | 100 | mg |
| | sodium hydroxide | 35 | mg |
| | mannitol | 1500 | mg |
| | water for inj. | 50 | mL |

The ambroxol is dissolved in water at its own pH or optionally at pH 5.5 to 6.5 and mannitol is added to make it isotonic. The solution obtained is filtered free from pyrogens and the filtrate is transferred under aseptic conditions into injection vials which are then sealed with rubber stoppers and autoclaved.

| F) | Suppositories | |
|---|---|---|
| | ambroxol | 450 mg |
| | solid fat | 1650 mg |

The hard fat is melted. At 40° C. ambroxol is homogeneously dispersed therein. The mixture is cooled to 38° C. and poured into slightly chilled suppository moulds.

| G) | Oral Solution | | |
|---|---|---|---|
| | ambroxol | 150 | mg |
| | hydroxyethylcellulose | 50 | mg |
| | sorbic acid | 5 | mg |
| | sorbitol (70%) | 600 | mg |
| | glycerol | 200 | mg |
| | flavoring | 15 | mg |
| | water | to 10 | mL |

Distilled water is heated to 70° C. Hydroxyethylcellulose is dissolved therein with stirring. After the addition of sorbitol solution and glycerol the mixture is cooled to ambient temperature. At ambient temperature, sorbic acid, flavoring, and ambroxol are added. To eliminate air from the suspension it is evacuated with stirring.

1. A method of treating acute pain in a patient in need thereof, the method comprising orally administering to the patient ambroxol or a pharmacologically acceptable salt thereof.

2. The method according to claim 1, wherein the acute pain is operative pain.

3. The method according to claim 1, wherein the acute pain is acute pain of herpes zoster.

4. The method according to claim 1, wherein the acute pain is toothache pain.

5. The method according to claim 1, wherein the acute pain is pain after trauma.

6. The method according to claim 1, wherein the acute pain is pain after burns.

7. The method according to claim 1, wherein the acute pain is pain after stroke or cardiac infarct.

8. The method according to claim 1, wherein the acute pain is pain in pancreatitis.

9. The method according to claim 1, wherein the acute pain is pain in colic.

10. The method according to claim 1, wherein the acute pain is pain in cramps.

11. The method according to one of claims 1 to 10, wherein the ambroxol or a pharmacologically acceptable salt thereof is administered to the patient in a daily dose of 30 mg to 4000 mg.

12. The method according to one of claims 1 to 10, wherein one or more additional active substances selected from NSAIDs, opioids, anticonvulsants, local anesthetics, antidepressants, and glutamate receptor antagonists are administered to the patient before, after, or simultaneously with the ambroxol or a pharmacologically acceptable salt thereof.

13. The method according to one of claims 1 to 10, wherein one or more additional active substances selected from calcium channel blockers, sodium channel blockers, nicotine agonists, and alpha-adrenergic agonists are administered to the patient before, after, or simultaneously with the ambroxol or a pharmacologically acceptable salt thereof.

14. A pharmaceutical composition comprising:
(a) ambroxol or a pharmacologically acceptable salt thereof; and
(b) one or more additional active substances selected from NSAIDs, opioids, anticonvulsants, local anesthetics, antidepressants, and glutamate receptor antagonists.

15. A pharmaceutical composition comprising:
(a) ambroxol or a pharmacologically acceptable salt thereof; and
(b) one or more additional active substances selected from calcium channel blockers, sodium channel blockers, nicotine agonists, and alpha-adrenergic agonists.
